(19) United States
(12) Patent Application Publication
(10) Pub. No.: US 2009/0105673 A1
(43) Pub. Date: Apr. 23, 2009

(54) MEDICAMENT APPLICATOR

(76) Inventors: Gregory R. Cascio, Chicago, IL (US); Jeffrey D. Bransky, Chicago, IL (US); James D. Morrow, Oak Park, IL (US)

Correspondence Address: NEAL, GERBER, & EISENBERG, SUITE 2200, 2 NORTH LASALLE STREET, CHICAGO, IL, 60602, US

(21) Appl. No.: 12/203,874

(22) Filed: Sep. 3, 2008

Related U.S. Application Data
(63) Continuation-in-part of application No. 11/873,884, filed on Oct. 17, 2007.

(51) Int. Cl. A61M 35/00; A61D 7/00

(52) U.S. Cl. 604/310

(57) ABSTRACT

A medicament applicator for use with a medicament cartridge is disclosed herein. The applicator comprises a first member and a second member, and a first brush member and a second brush member. Moving the first member towards the second member causes the first brush member to move away from the second brush member, and causes the medicament cartridge to open, allowing its contents to exit.

### CROSS REFERENCE TO RELATED APPLICATION

This application is a continuation-in-part of U.S. application Ser. No. 11/873,884, entitled “Medicament Applicator”, which was filed on Oct. 17, 2007, which is incorporated herein by reference in its entirety.

### FIELD OF THE INVENTION

This invention relates to a medicament applicator, such as for use with animals and such as for applying the medicament to skin.

### SUMMARY OF THE IN INVENTION

A medicament applicator is described herein. The details of the invention are set forth below in connection with the detailed description of the embodiments.

### BRIEF DESCRIPTION OF THE DRAWINGS

FIG. 1 is a perspective view of a first embodiment of the present invention.

FIG. 2 is a perspective view of a second embodiment of the present invention, including a medicament cartridge.

FIG. 3 is a side view of an exemplary medicament cartridge.

FIG. 4 is a front view of the exemplary medicament cartridge set forth in FIG. 3.

FIG. 5 is a perspective view of a third embodiment of the present invention.

FIG. 6 is a side view of a fourth embodiment of the present invention, including a medicament cartridge.

FIG. 7 is a side view of the fourth embodiment of the present invention of FIG. 6, where the medicament cartridge has been opened.

FIG. 8 is a top view of an exemplary changeable head.

FIG. 9 is a perspective view of a fifth embodiment of the present invention in use and in the first, unopened position.

FIG. 10 is a perspective view of the exemplary embodiment as seen in FIG. 9, in use and in the second, opened position.

FIG. 11 is a side view of a sixth embodiment of the present invention in the first, unopened position.

FIG. 12 is a side view of the sixth embodiment as seen in FIG. 11, in the second, opened position.

FIG. 13 is a side view of a seventh embodiment of the present invention in the first, unopened position.

FIG. 14 is a side view of the seventh embodiment of FIG. 13, in the second, opened position.

FIG. 15 is a side view of an eighth embodiment of the present invention in the first, unopened position.

FIG. 16 is a side view of the eighth embodiment as seen in FIG. 15, in the second, opened position.

FIG. 17 is a side view of the eighth embodiment as seen in FIG. 15, returned to the first, unopened position.

FIG. 18 is a side view of the ninth embodiment of the present invention.

FIG. 19 is a close up of the separating apparatus of the embodiment depicted in FIG. 18.

FIG. 20 is a perspective view of a tenth embodiment of the present invention.

FIG. 21 is a front-end view of an eleventh embodiment of the present invention.

FIG. 22 is a side view of the eleventh embodiment as seen in FIG. 21.

FIG. 23a-e are front, top, back, bottom and side views, respectively, of the twelfth embodiment of the present invention.

FIG. 24 is a side view of the twelfth embodiment as seen in FIG. 23, in a second position.

FIG. 25 is a cross sectional view of the twelfth embodiment as seen in FIG. 24.

FIG. 26 is an exploded view of the twelfth embodiment as seen in FIG. 23.

FIG. 27 is a side view of the thirteenth embodiment of the present invention.

FIG. 28 is a top view of the thirteenth embodiment as seen in FIG. 27.

FIG. 29 is a cross-sectional view of the thirteenth embodiment as seen in FIG. 27, partially between a first position and a second position.

FIG. 30 is a cross sectional view of the thirteenth embodiment as seen in FIG. 27, in a first position.

FIG. 31 is a cross sectional view of the thirteenth embodiment as seen in FIG. 27, in a second position.

### DETAILED DESCRIPTION OF THE DRAWINGS

The present invention is described herein with respect to exemplary medicament applicators that dispense medicament directly to a substrate without requiring the user to touch the medicament. It will be understood that various other applicators can be used in accordance with the present invention. By way of example, and in no way limiting, one use of the present invention would be to expose the skin of an animal by temporarily displacing the fur of the animal, in order to dispense medicament, such as flea or tick medicine, directly to the skin of an animal.

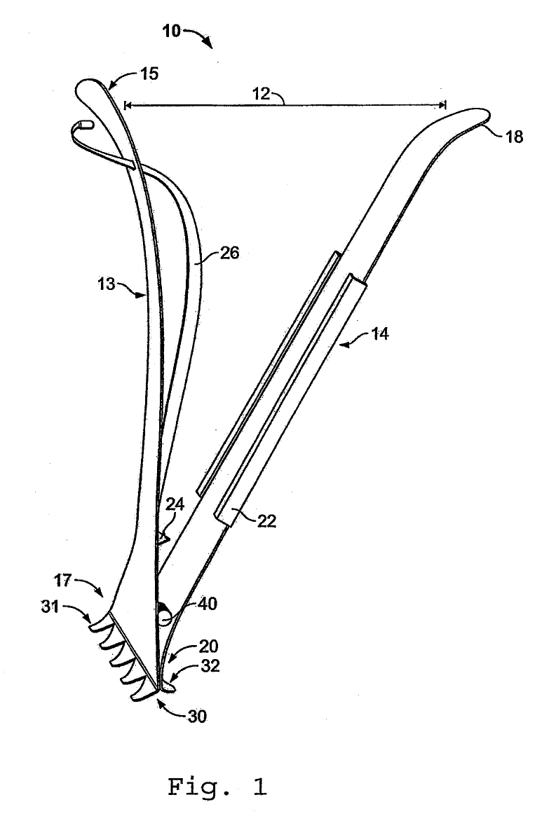

In the embodiment depicted in FIG. 1, applicator 10 comprises handle apparatus 12, which comprises first handle member 13 and second handle member 14. First handle member 13 comprises first end 15, which forms a first gripping portion 16, and second end 17, opposite first end 15. Second handle member 14 comprises first end 18, which forms second gripping portion 19, and second end 20, opposite first end 18.

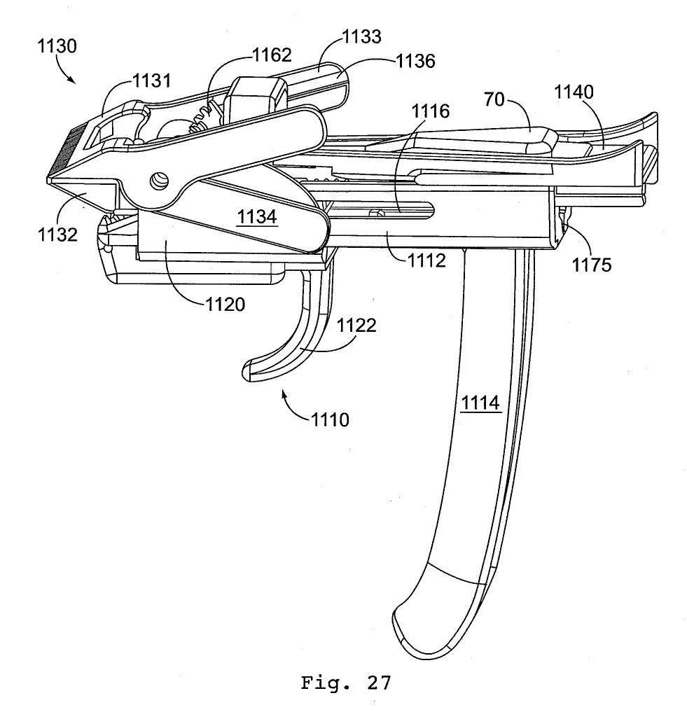

In order to facilitate the parting of hair, fur, or other material above the substrate, applicator 10 further comprises separating apparatus 30. In the first embodiment, separating apparatus 30 comprises first hair comb 31, which is integrally formed from first handle member 13, and second hair comb 32, which is integrally formed from second handle member 14. Separating apparatus 30 is movable between a first, unopened separating position (shown in FIG. 27), and a second, opened separating position. As described more fully below, the first and second separating positions correspond to first and second handle member positions.

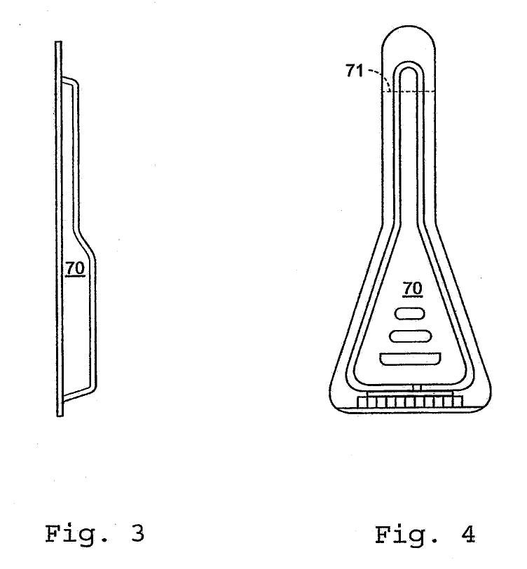

As stated above, applicator 10 can be used with a medicament cartridge, such as cartridge 70, shown in FIGS. 3 and 4. In order to better facilitate the use of such a cartridge, applicator 10 further comprises retainer 22, opening apparatus 24 and compressing apparatus 26. Retainer 22 is formed on second handle member 14. In use, a medicament cartridge (not shown) is placed in, and secured by, retainer 22. In the depicted embodiment, retainer 22 is integrally formed from second handle member 14, but it will be apparent to those in the art that the scope of the present invention includes an embodiment in which retainer 22 is removably secured to second handle member 14.

To aid in the opening of a cartridge, such as medicament cartridge 70, opening apparatus 24 is provided and secured to first handle member 13. In use, opening apparatus 24 engages a medicament cartridge (not shown) and thus opens the cartridge. In the depicted embodiment, opening apparatus 24 is integrally formed from first handle member 13. However, it will be apparent to those in the art that the scope of the present invention includes those embodiments in which the opening apparatus is otherwise secured to first handle member 13. Moreover, it will be apparent that the scope of the present invention further includes those embodiments in which the opening apparatus is secured to other elements of apparatus 10, so long as it opens a cartridge when apparatus 10 is used.

As seen in FIG. 1, opening apparatus 24 opens the cartridge by piercing it. As described more fully below, the scope of the present invention includes those embodiments in which opening apparatus 24 opens the cartridge in any number of ways, including cracking the cartridge, pushing a tip of the cartridge, etc.

To aid in dispensing the medicament, compressing apparatus 26 is provided and secured to first handle member 15. In the embodiment depicted in FIG. 1, compressing apparatus 26 is a tensioned spring, either metal or plastic, which engages a medicament cartridge when the handle members are moved to the second position as described more fully below.

Applicator 10 further comprises hinge member 40. Hinges, such as hinge member 40, are well known throughout the art, and will not be described in detail herein. As seen, hinge member 40 connects handle member 13 and handle member 14 such that they are rotatable with respect to one another between a first handle position and a second handle position. In the first handle position, depicted in FIG. 1, first ends 15 and 18 of respective handle members 13 and 14, are apart, while second ends 17 and 20 are together. In the second handle position (not shown), first ends 15 and 18 are together, while second ends 17 and 20 are separated. It will be appreciated that applicator 10 may further comprise a biasing spring (not shown) to bias the handle members to the first handle position.

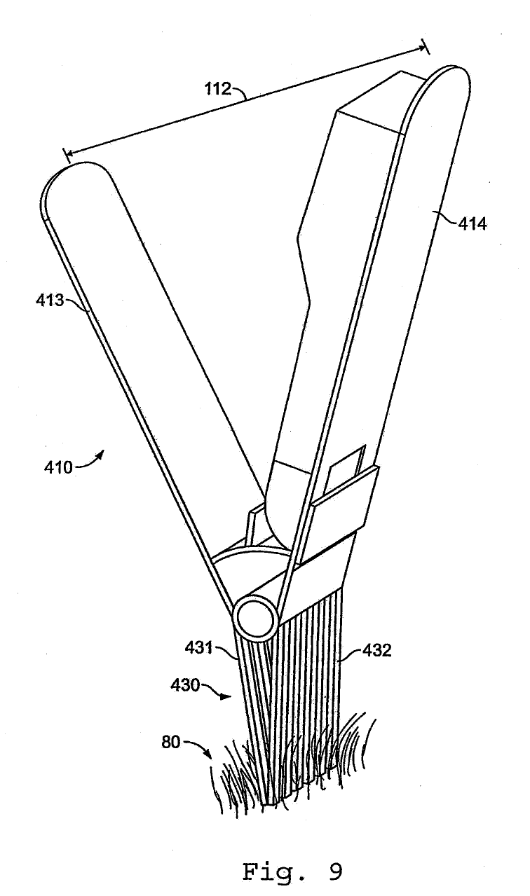

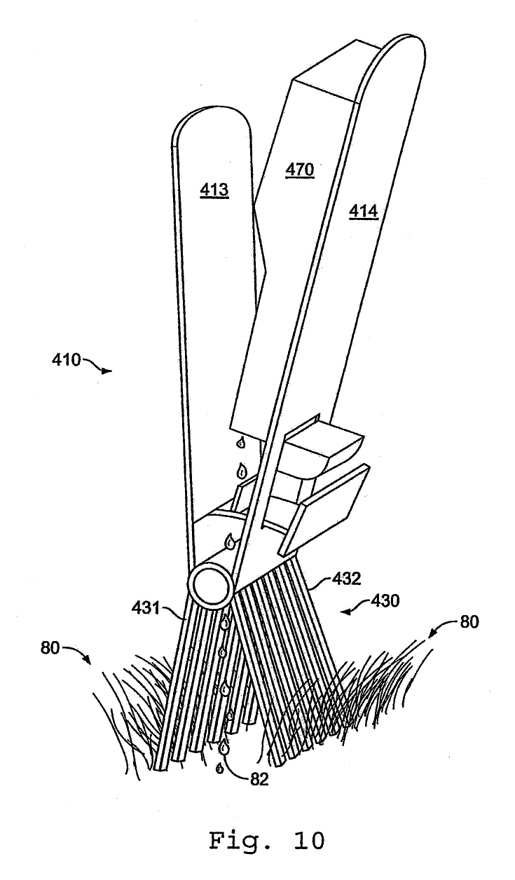

Applicator 10 is used in a similar way as the embodiment depicted in FIGS. 9 and 10. While the embodiment in FIGS. 9 and 10 differs from the first embodiment, the use of the embodiments is similar, and therefore, FIGS. 9 and 10 can be used as a reference.

In use, applicator 10 works as follows. The user places a medicament cartridge in retainer 22. The user next places both brushes 31 and 32 of separating apparatus 30 on an animal, a person's head, or any other object that comprises a substrate to which the medicament must be applied. The user then compresses handle members 13 and 14, moving them from the first handle position to the second handle position. This causes hair brushes 31 and 32 to separate, parting any hair engaged to or proximate the hair brushes 31 and 32, and exposing the skin (or other substrate) underneath. Moving handle members 13 and 14 to the second handle position also causes compressing apparatus 26 to engage the medicament cartridge, and opening member 24 to engage and open the medicament cartridge. In this way, once the skin (or other substrate) has been exposed, the medicament contained in the cartridge can be expelled directly to the skin. It will be appreciated by those in the art that the scope of the present invention includes an embodiment in which a compressing apparatus, such as compressing apparatus 26, first engages a medicament cartridge, then a user selectively compresses the compressing apparatus, thus expelling the medicament.

The remaining embodiments each comprise similar elements as those comprised by applicator 10. As such, each embodiment will only briefly be described herein. Furthermore, the elements that are similar among the embodiments have similar reference numerals. By way of example, element 15 of applicator 10 will be similar to element 115 of applicator 110, element 215 of applicator 210, etc.

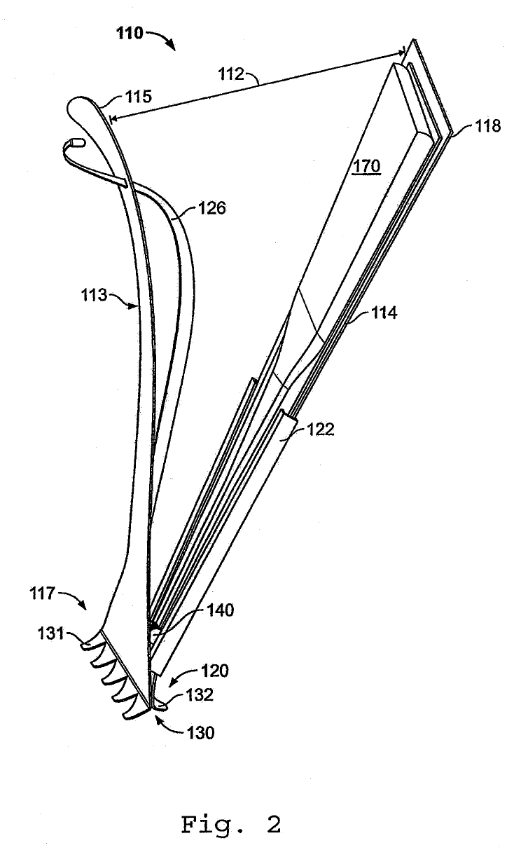

FIG. 2 depicts a second embodiment of the present invention. As seen, applicator 110 closely resembles applicator 10 depicted in FIG. 1, and comprises many similar elements. By way of example, applicator 110 comprises handle apparatus 112, which comprises first handle member 113 and second handle member 114. Separating apparatus 130 comprises first hair brush 131 and second hair brush 132, both of which are engaged to second ends 117 and 120, respectively, of handle members 113 and 114, respectively. Applicator 110 further comprises compressing apparatus 126 engaged to first handle member 113, and retainer 122 engaged to second handle member 114. Applicator 110 further comprises medicament cartridge 170. As seen, medicament cartridge 170 is engaged to, and retained by, retainer 122. In this manner, applicator 110 may be used repeatedly, and only cartridge 170 must be replaced after each application.

In each of the above embodiments, and in each of the embodiments that are described below, the medicament cartridge is removably secured to the applicator in some manner. Because of this, the same applicator can be repeatedly used, and only a new cartridge need be purchased or employed between uses. It will be appreciated by those in the art, however, that the scope of the present invention includes those embodiments in which a medicament applicator is integrally formed on an applicator. In those embodiments, the entire applicator may be disposed after an application.

FIGS. 3 and 4 depict an exemplary medicament cartridge 70. It will be appreciated by those in the art that the scope of the present invention is not limited to the shape of cartridge 70. Moreover, as described more fully below, cartridge 70 is opened by cracking seal 71. However, it will be appreciated by those in the art that the scope of the present invention includes any method of opening a medicament cartridge such as cartridge 70.

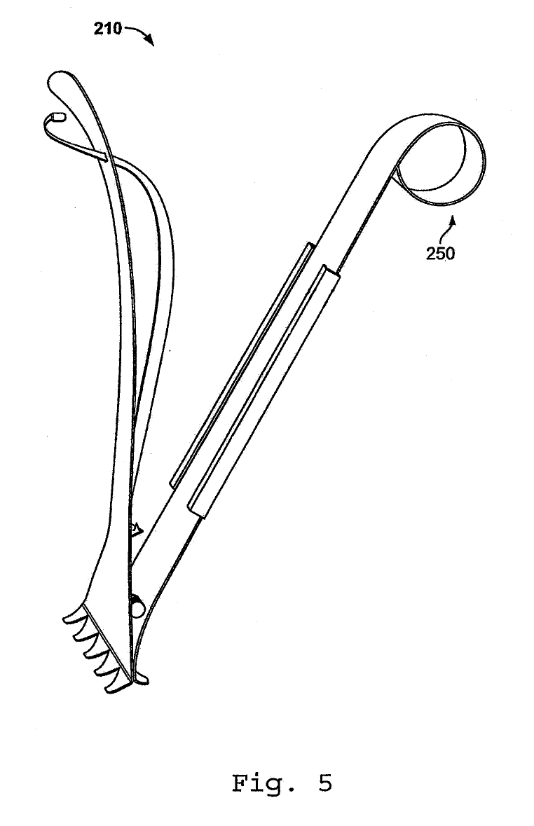

A third embodiment of the present invention is depicted in FIG. 5. Applicator 210 is nearly identical to applicator 10 depicted in FIG. 1. However, in order to provide a grip for the user, a finger loop 250 is formed on second end 218 of second handle member 214.

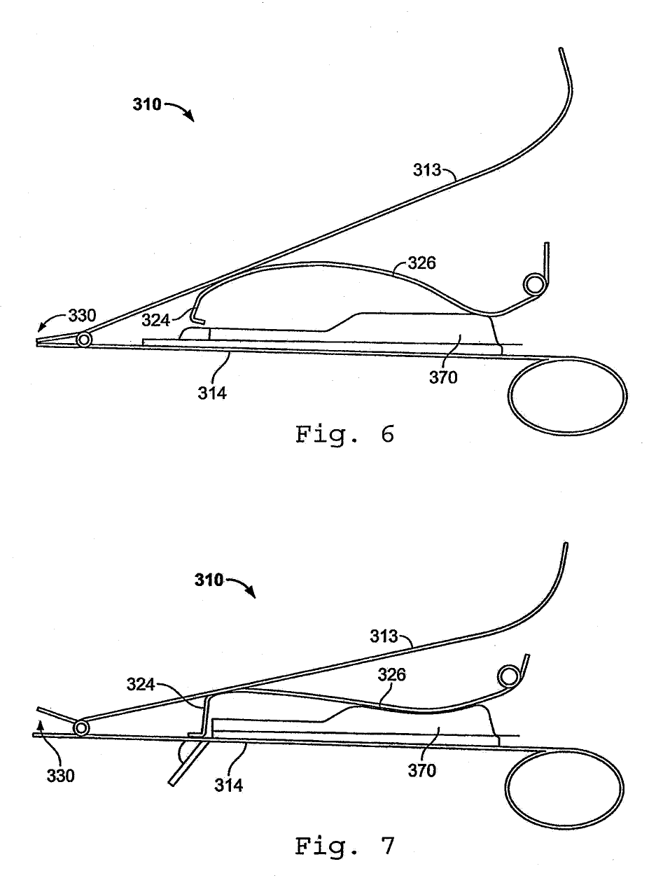

FIGS. 6 and 7 depict a fourth embodiment of the present invention. As seen, applicator 310 comprises similar elements to those disclosed in the previous embodiments. By way of example, and in no way limiting, applicator 310 comprises first handle member 313, second handle member 314, separating apparatus 330, and medicament cartridge 370. In the embodiment depicted in FIGS. 6 and 7, compressing apparatus 326 and opening apparatus 324 are formed as a single element. In the first handle position, depicted in FIG. 6, opening apparatus 324 is not engaged to medicament cartridge 370. While compressing apparatus 326 is shown engaged to cartridge 370, it will be understood by those in the art that compressing apparatus 326 is comprised of flexible metal or plastic and, as such, can be disengaged from cartridge 370 temporarily in order to install or remove cartridge 370 from applicator 310.

When handle members 313 and 314 are moved to the second handle position, as depicted in FIG. 7, opening apparatus 324 engages and opens cartridge 370. As seen, opening apparatus 324 opens cartridge 370 by “cracking” a seal formed on cartridge 370. It will be appreciated by the art that the scope of the present invention includes any means by which opening apparatus 324 can engage and open medicament cartridge 370. Also shown in FIG. 7, compressing apparatus 326 fully engages, and deforms medicament cartridge 370, expelling the medicament from cartridge 370.

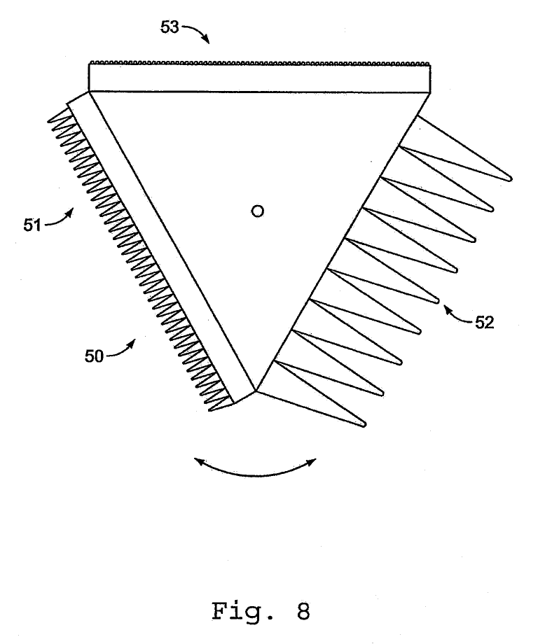

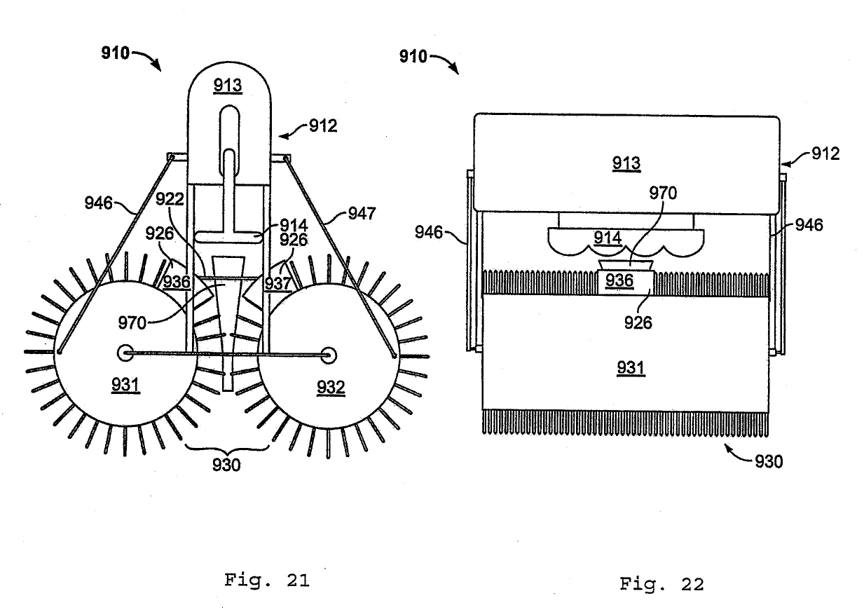

It may be desirable to provide an embodiment of the present invention with a means by which the applicator can accommodate different hair lengths using the same basic structure. To that end, FIG. 8 depicts an embodiment of a hair brush similar to hair brushes 31 and 32 depicted in FIG. 1. Hair brush 50 can be used with any embodiment described herein, except the embodiment depicted in FIGS. 21 and 22, which are described below. As seen, hair brush 50 has a first side 51, a second side 52 and a third side 53. Hair brush 50 is rotatably engaged to any of the embodiments, and each side 51, 52 and 53 has a different comb-tooth length. In this manner, a user can use the same applicator for various animals with differing hair lengths. By way of example, and in no way limiting, a user can use an applicator with hair brush 50 on a short-haired dog, such as a Chihuahua, by rotating hair brush 50 so that third side 53 engages the hair of the animal. The user can then use the same applicator on a long-haired dog, such as a German Shepherd, by rotating hair brush 50 so that second side 52 engages the hair of the animal.

In a second embodiment of an accommodating hair brush, a basic applicator, such as the applicator shown in FIG. 1, may be coupled with a removable guard, or a plurality of removable guards. A pair of base hair brushes, similar to hair brushes 31 and 32, would be formed on the applicator. The base hair brushes would be similar in length and use as side 51 of the embodiment depicted in FIG. 8. In other words, the base hair brushes would be ideal for use with very short hair. The removable guard, or each of a plurality of guards, would then slide over the base hair brushes and lock in place. If one removable guard is used, then it would allow the applicator to be used with hair that is longer than would be ideal with the base hair brushes. If a plurality of guards is used, then each guard would allow the applicator to be used with different hair lengths. The lock could be any one of a known manner, and may include a tongue-in-groove mechanism, slot and tab mechanism, etc.

FIGS. 9 and 10 depict a fifth embodiment of the present invention, and show the invention in use. As seen, an exemplary use of the present invention, but by no way a limiting use, is allowing the user to dispense a medicament, such as a flea or tick medicine, directly to the skin of an animal, without touching the medicament. To that end, applicator 410 is shown in use on an animal (not shown) having hair 80. In the first handle position, depicted in FIG. 9, hair brushes 431 and 432 of separating apparatus 430 are closed and placed in hair 80, proximate the skin of the animal (not shown). Once first handle 413 and second handle 414 of handle apparatus 412 are moved to the second handle position, depicted in FIG. 10, medicament cartridge 470 is opened, and simultaneously, hair 80 is parted, exposing the skin of the animal. This allows medicament 82 to be delivered directly to the skin of the animal.

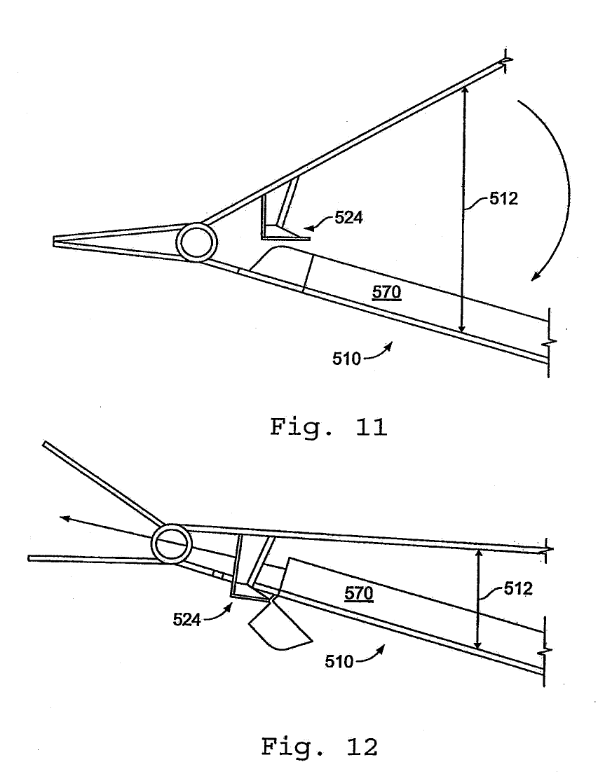

FIGS. 11 through 17 depict embodiments of the present invention employing differing embodiments of an opening apparatus similar to the opening apparatuses described above. FIGS. 11 and 12 depict opening apparatus 524, which engages and cracks a seal formed on cartridge 570 when handle apparatus 512 is moved from the first handle position, depicted in FIG. 11, to the second handle position, depicted in FIG. 12.

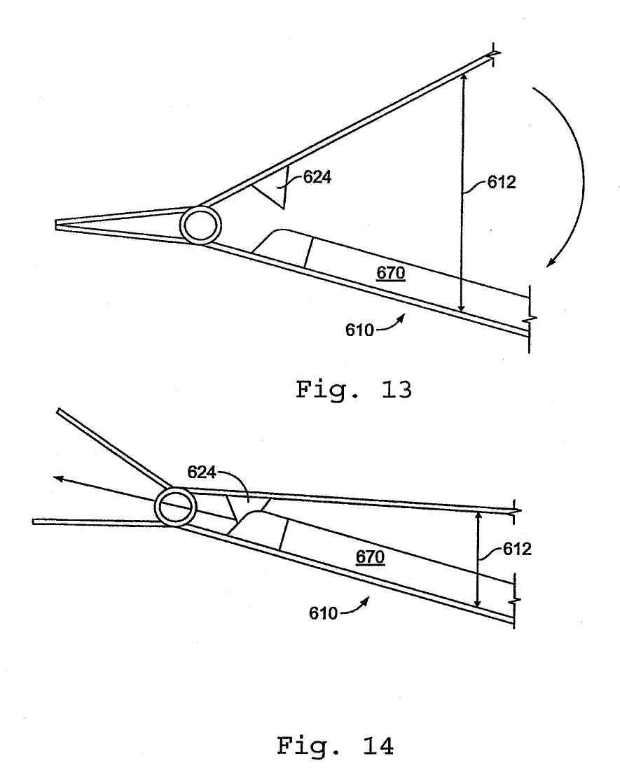

FIGS. 13 and 14 depict applicator 610, which comprises opening apparatus 624. As seen, opening apparatus 624 engages and pierces medicament cartridge 670 when handle apparatus 612 is moved from the first handle position, depicted in FIG. 13, to the second handle position, depicted in FIG. 14.

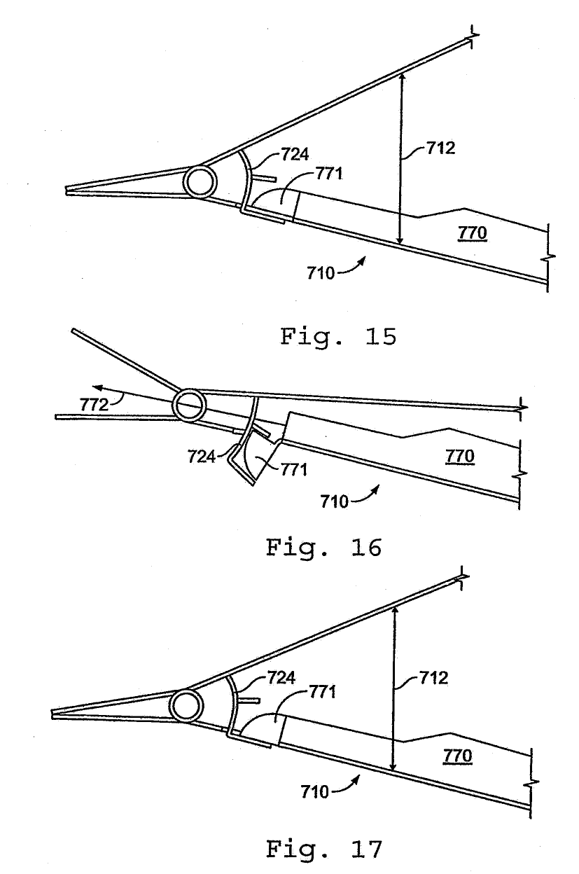

FIGS. 15 through 17 depict applicator 710, and the progression of opening apparatus 724 in use. As seen, FIG. 15 depicts handle apparatus 712 in the first handle position. When handle apparatus 712 is moved to the second handle position, depicted in FIG. 16, opening apparatus 724 engages tip 771 of cartridge 770, thus opening cartridge 770. This allows medicament (not shown) to flow from cartridge 770 along line 772 to the intended target, such as the skin of an animal, or other substrate. When handle apparatus 712 is returned to the first handle position, as depicted in FIG. 17, opening apparatus 724 again engages tip 771, moving it to the position depicted in FIG. 17, thus closing cartridge 770, preventing medicament from flowing from cartridge 770.

It will be appreciated by those in the art that any of the opening apparatuses depicted in FIGS. 11 through 17 may be used with any applicator embodiment disclosed herein, except for the embodiment disclosed in FIGS. 21 and 22. It will likewise be appreciated by those in the art that any compressing apparatus disclosed in any of FIGS. 1 through 20 may be used with any embodiment of an applicator disclosed herein, except for the embodiment disclosed in FIGS. 21 and 22.

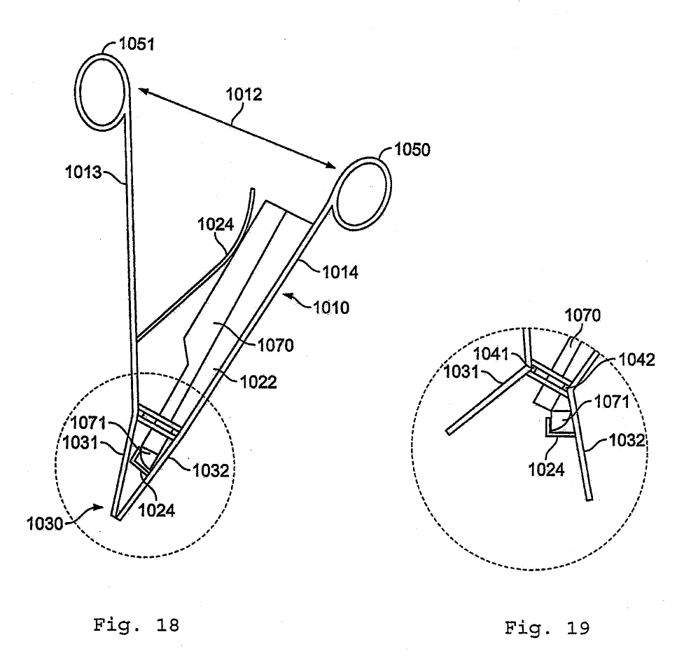

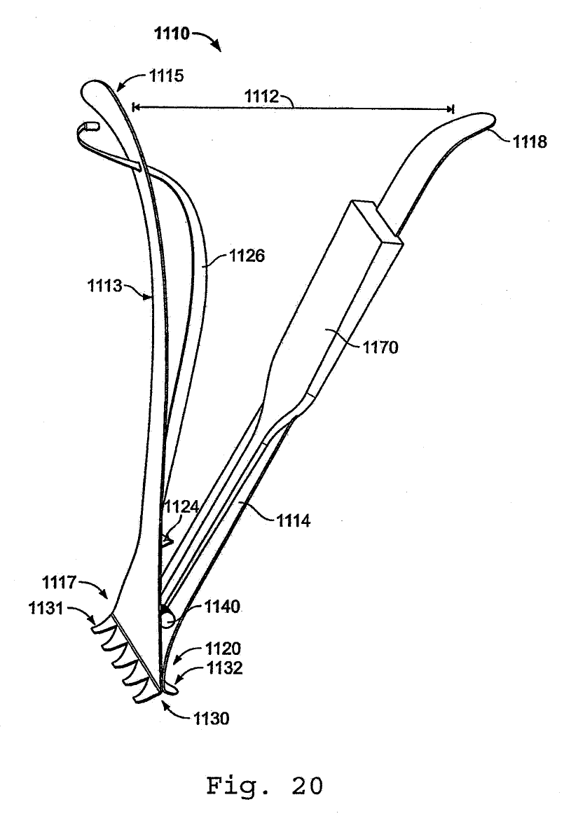

FIG. 18 depicts a ninth embodiment of the present invention. As seen, the ninth embodiment has several features in common with the previous embodiments. For example, applicator 1010 comprises handle apparatus 1012, which comprises first handle member 1013 and second handle member 1014, compressing apparatus 1026, retainer 1022, and opening apparatus 1024, which cracks medicament cartridge 1070, as described above. As seen, retainer 1022 is slightly raised such that medicament cartridge 1070 is not directly engaged to handle member 1014. In this manner, tip 1071 of medicament cartridge 1070, when closed as in Fig. Y, is centered between handle members 1013 and 1014. Handle apparatus 1012 is movable between a first handle position, depicted in FIG. 18, and a second handle position, depicted in FIG. 19.

The embodiment depicted in FIG. 19 comprises a hinge member 1040. However, unlike the previous embodiments, hinge member 1040 comprises two hinges, 1041 and 1042. In this manner, medicament cartridge 1070 may extend through hinge member 1040.

FIG. 20 depicts a tenth embodiment of the present invention, which is similar to the embodiments of FIGS. 1 and 2. As seen in FIG. 20, in applicator 1110, cartridge 1170 is integrally formed with second handle member 1114. It will be appreciated by those in the art that, like the embodiment depicted in FIG. 20, the present invention contemplates that in any of the embodiments depicted herein, the medicament cartridge may be removably secured to the applicator, or the medicament cartridge may be integrally formed from/with the applicator.

FIGS. 21 and 22 depict an eleventh embodiment of the present invention. As seen, applicator 910 comprises nearly the same elements as the above-described embodiments. By way of example, applicator 910 comprises handle apparatus 912, which comprises a first handle member 913 and a second handle member 914. Handle apparatus 912 is movable between a first handle position, depicted in FIG. 21, and a second handle position, as described more fully below.

As seen, applicator 910 further comprises separating apparatus 930. In the depicted embodiment, separating apparatus 930 comprises first hair comb 931, and second hair comb 932. In applicator 910, each hair comb 931 and 932 is a rotating brush. Additionally, each rotating brush 931 and 932 can rotate between a first brush position, depicted in FIG. 21, and a second brush position. As described more fully below, and as will be apparent to those in the art, rotating brush 931 rotates in the opposite direction from rotating brush 931. Moreover, the first and second brush positions correspond to first and second handle member positions.

As seen, each hair brush 931 and 932 is engaged to second handle member 914 by way of a pair of struts 946 and a pair of struts 947, respectively. As seen, a first strut of struts 946 engages the front side of brush 931, as shown in FIG. 21. Likewise, the second strut of struts 946 engages the rear side of brush 931, the side opposite that shown in FIG. 21. Struts 947 engage brush 932 in a similar manner. In this manner, movement of second handle member 914 with respect to first handle member 913 causes rotation of each hair brush 931 and 932 as described more fully below.

Applicator 910, like the previously described embodiments, can be used with a medicament cartridge, such as cartridge 970. In order to better facilitate the use of such a cartridge, applicator 910 further comprises retainer 922 and compressing apparatus 926. Retainer 22 is formed on frame 927 of applicator 910. As seen, medicament cartridge 970 is placed in, and secured by, retainer 922.

As stated above, applicator 910 further comprises compressing apparatus 926. Compressing apparatus 926 comprises first compressor 936, which is formed on first hair brush 931, and second compressor 937, which is formed on second hair brush 932. In the embodiment depicted in FIGS. 21 and 22, each compressor 936 and 937 rotates with each respective hair brush 931 and 932. In this manner, as separating apparatus 930 moves from the first brush position to the second brush position, compressors 936 and 937 engage medicament cartridge 970.

In use, applicator 910 works in a similar fashion to the applicators described above. The user places medicament cartridge 970 in retainer 922. The user next places both brushes 931 and 932 of separating apparatus 930 on, for example, an animal. The user then compresses handle members 913 and 914, moving them from the first handle position, depicted in FIG. 21, to the second handle position. The movement of second handle member 914 causes hair brushes 931 and 932 to rotate, parting any hair engaged to the hair brushes and exposing the skin (or other substrate) underneath. In the depicted embodiment, movement of second handle member 914 up causes hair brush 931 to rotate clockwise and brush 932 to move counter-clockwise, as depicted in FIG. 21. Moving handle members 913 and 914 to the second handle position also causes compressing apparatus 926 to engage medicament cartridge 970, compressing cartridge 970 and expelling any medicament from cartridge 970.

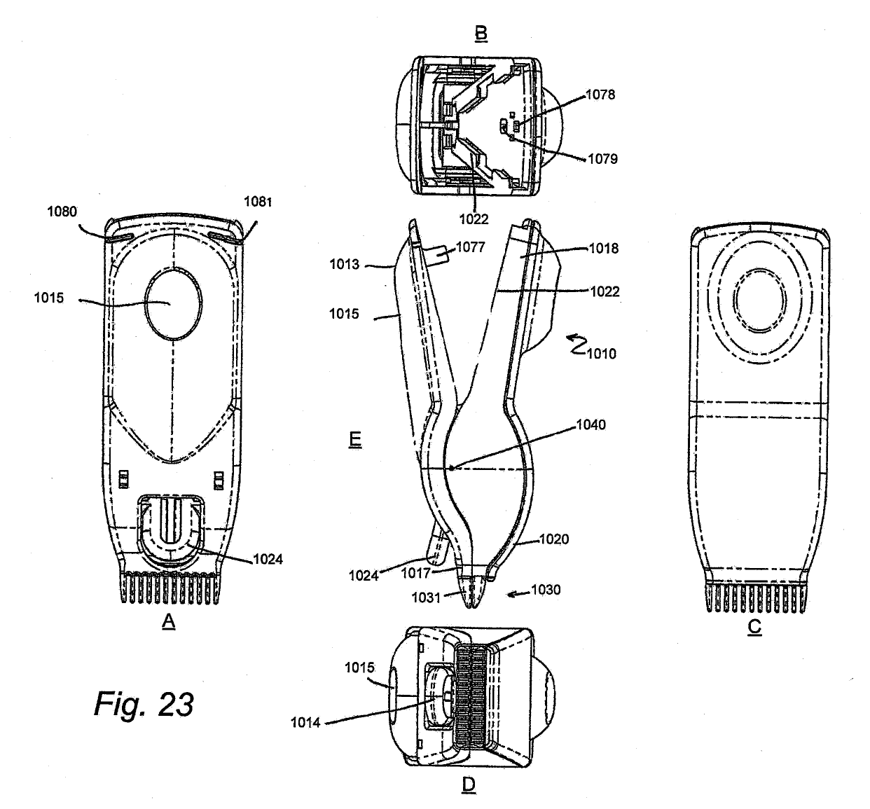

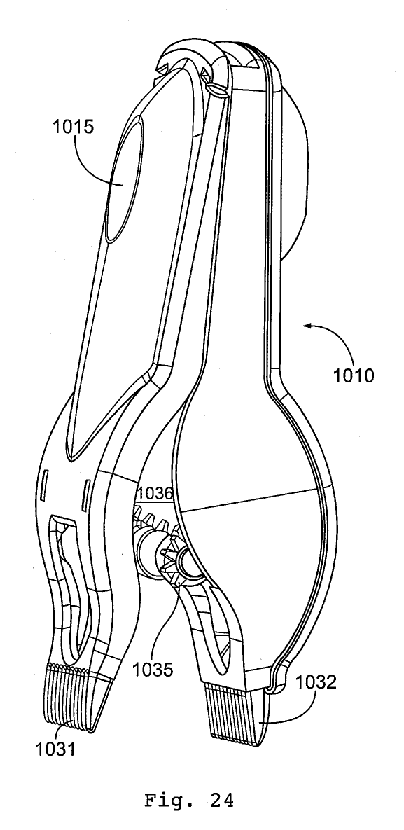

FIGS. 23-26 depict a twelfth embodiment of the present invention. As seen in FIG. 23, applicator 1010 comprises first handle member 1013 and second handle member 1014. First handle member 1013 comprises first end 1015, which forms a first gripping portion 1016, and second end 1017, opposite first end 1015. Second handle member 1014 comprises first end 1018, which forms second gripping portion 1019, and second end 1020, opposite first end 1018.

In order to facilitate the parting of hair, fur, or other material above the substrate, applicator 1010 further comprises separating apparatus 1030. In the embodiment depicted for example in FIG. 23, separating apparatus 1030 comprises first hair comb 1031, which is integrally formed from first handle member 1013, and second hair comb 1032, which is integrally formed from second handle member 1014. Separating apparatus 1030 is movable between a first, unopened separating position (shown in FIG. 23), and a second, opened separating position (shown in FIG. 24). As described more fully below, the first and second separating positions correspond to first and second handle member positions.

As with the previously described embodiments, applicator 1010 can be used with a medicament cartridge, such as cartridge 70 described above. In order to better facilitate the use of such a cartridge, applicator 1010 further comprises retainer 1022, opening apparatus 1024 and piercing apparatus 1026. Retainer 1022 is formed on second handle member 1014. In use, medicament cartridge 70 is placed in, and secured by, retainer 1022. In the depicted embodiment, retainer 1022 is integrally formed from second handle member 1014, but it will be apparent to those in the art that the scope of the present invention includes an embodiment in which retainer 1022 is removably secured to second handle member 1014.

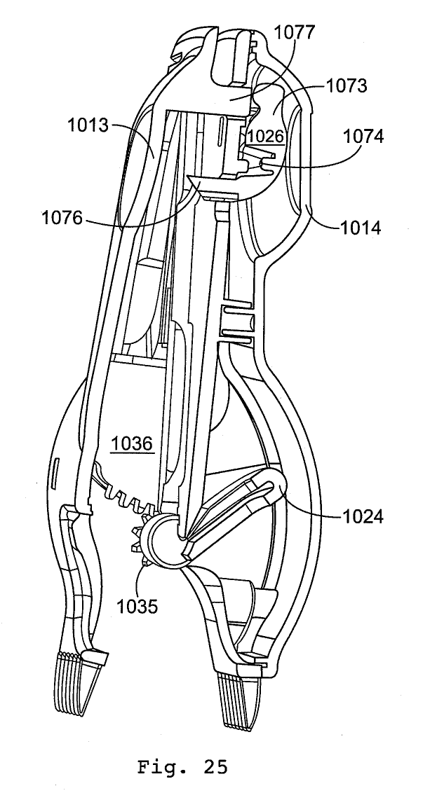

To aid in the use of medicament cartridge 70, opening apparatus 1024 is provided. As seen, opening apparatus 1024 is positioned proximate to cracking seal 71 of medicament cartridge 70. Opening apparatus 1024 is rotatably engaged to second handle 1014, and is rotatable between a first position (shown in FIG. 23), in which opening apparatus 1024 is coplanar with retainer 1022, and a second position (shown in FIGS. 24 and 25). As described in detail below, as opening apparatus 1024 is rotated to the second position, cartridge 70 is opened along cracking seal 71, allowing the medicament to exit the cartridge 70.

A first partial-gear pair 1035 is formed on opening apparatus 1024 and is engaged to a second partial-gear pair 1036, which is integrally formed with first handle member 1013. It will be understood by those of skill in the art that, while the depicted embodiment has a first and second pair of partial-gears, the scope of the present invention includes those embodiments in which only one partial gear is formed on opening apparatus 1024 and a second partial gear is formed from first handle member 1013. It will further be understood by those in the art that the scope of the present invention includes those embodiments in which the partial gears 1035 and 1036 may be full gears.

To aid in dispensing the medicament, piercing apparatus 1026 is provided and secured to second handle member 1014. In the embodiment depicted, for example, in FIG. 25, piercing apparatus 1026 comprises arm 1073 that is pivotally engaged to second handle member 1014. Arm 1073 comprises a first protrusion 1075 and a second protrusion 1076. Ann 1073 pivots about pivot point 1074 between a first position, in which second protrusion 1076 does not extend through second opening 1079 formed in second handle member 1014, and a second position, in which second protrusion 1076 does extend through second opening 1079. As seen, first protrusion 1075 is formed on the opposite side of pivot point 1074 from second protrusion 1076. Piercing apparatus 1026 may further include a first biasing spring 1085 that biases piercing apparatus 1026 to its first position.

When the handle members 1013 and 1014 are moved to the second position as described in detail below, extension 1077, integrally formed from first handle member 1013, engages first protrusion 1075 through first opening 1078 formed in second handle member 1014. The engagement of extension 1077 to first protrusion 1075 forces arm 1073 into its second position, causing second protrusion 1076 to extend through second opening 1079. As second protrusion 1076 extends through second opening 1079, it pierces cartridge 70, creating an air hole. This removes any vacuum effect within cartridge 70 and allows the medicament to smoothly and more quickly exit cartridge 70.

Applicator 1010 further comprises hinge member 1040. Hinges, such as hinge member 1040, are well known throughout the art, and will not be described in detail herein. As seen, hinge member 1040 connects first handle member 1013 and second handle member 1014 such that they are rotatable with respect to one another between a first handle position and a second handle position. Applicator 1010 may further comprise a second biasing spring (not shown) to bias the handle members to the first handle position.

In the first handle position, depicted in FIG. 23, first ends 1015 and 1018 of respective handle members 1013 and 1014, are apart, while second ends 1017 and 1020 are together. In the second handle position depicted in FIG. 25, first ends 1015 and 1018 are together, while second ends 1017 and 1020 are separated.

As is known in the art, medicament cartridges such as cartridge 70 typically contain animal medication that can be harmful if applied to a human's skin. As such, each cartridge is shipped in a foil wrapping that is intentionally difficult to open. In order to aid the user in opening the foil wrapping, package openers 1080 and 1081 may be provided. In the depicted embodiment, package openers 1080 and 1081 comprise two slots formed on first handle member 1013.

In the depicted embodiment, package opener 1080 is 8 mm long, while package opener 1081 is 10.5 mm long. In this manner, package opener 1080 may be used as a “wrench” side to break the plastic tab of the foil wrapping free, then package opener 1081 may be used to push through the foil wrapping to break it free from the case and allow the user to grab the foil and remove it from the plastic case. In the case of smaller pipettes or smaller packaging, package opener 1080 may also be used to push through the foil wrapping.

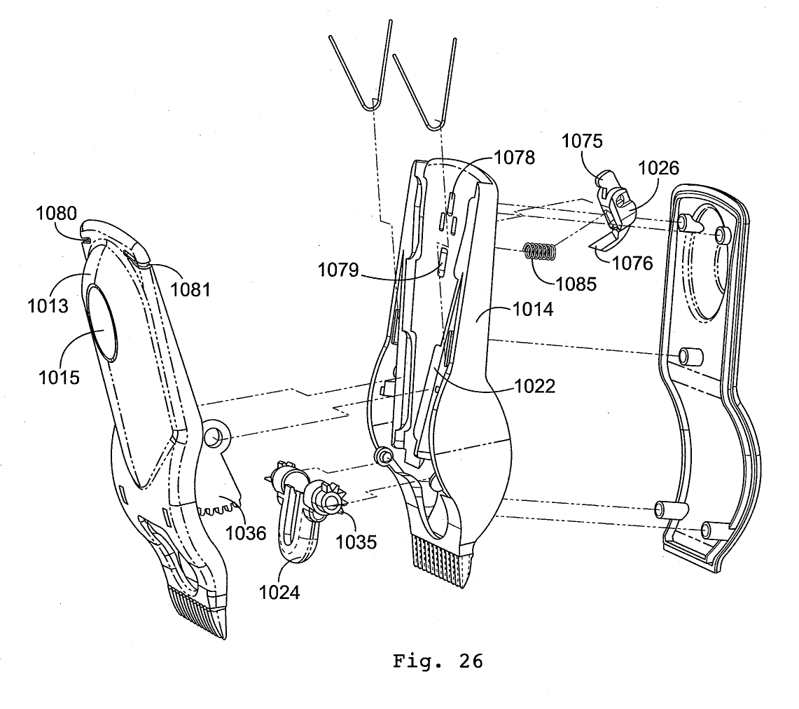

A stepped version, such as depicted in FIG. 26, may also be provided, where a step stops the package from being inserted into the slot at the 8 mm mark to break the tab free from the package. Once this is accomplished, the tab can be further slide into the 10.5 mm length to push through the foil wrapping.

In use, applicator 1010 works as follows. The user places medicament cartridge 70 in retainer 1022. The user next places both brushes 1031 and 1032 of separating apparatus 1030 on an animal, a person's head, or any other object that comprises a substrate to which the medicament must be applied. The user then compresses handle members 1013 and 1014, moving them from the first handle position to the second handle position. This causes hair brushes 1031 and 1032 to separate, parting any hair engaged to or proximate the hair brushes 1031 and 1032, and exposing the skin (or other substrate) underneath. Moving handle members 1013 and 1014 to the second handle position also causes extension 1077 to engage piercing apparatus 1026, moving piercing apparatus about pivot point 1074 to the second position as described above. Finally, opening member 1024, through partial gears 1035 and 1036, moves to its second position and opens medicament cartridge 70 along crack line 71. In this way, once the skin (or other substrate) has been exposed, the medicament contained in the cartridge can be expelled directly to the skin.

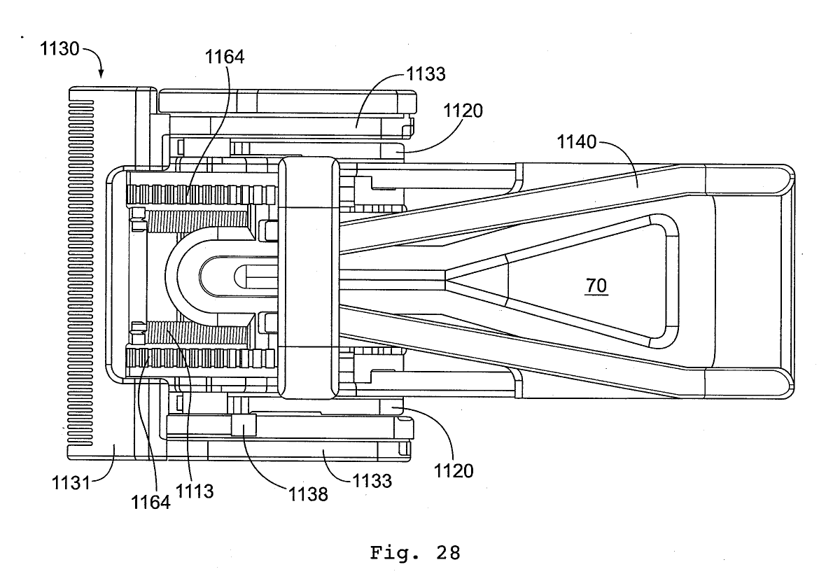

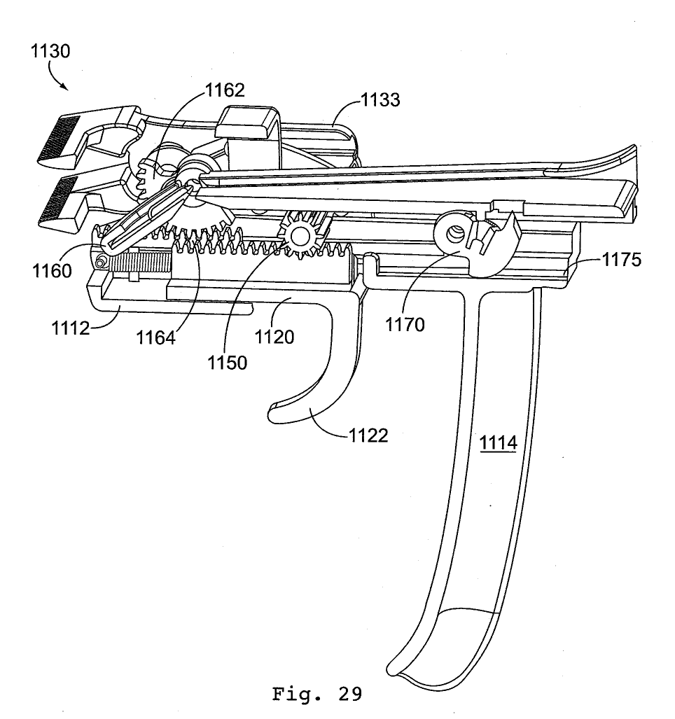

FIGS. 27 to 31 depict applicator 1110, a thirteenth embodiment of the present invention. As seen, applicator 1110 comprises base 1112 and handle 1114, which is integrally formed from base 1112. Trigger mechanism 1120 is engaged to base 1112 and is slidable between a first position, shown for example in FIG. 27, and a second position. Trigger 1122 extends and is integrally formed from trigger mechanism 1120. To aid the user in sliding trigger mechanism 1120 between its first and second positions, a plurality of guides (not shown) extend from trigger mechanism 1120 and engage slots 1116 formed on base 1112. As seen, for example, in FIG. 28, a plurality of biasing springs 1113 are connected to both base 1112 and trigger mechanism 1120 and bias trigger mechanism 1120 to its first position. It will be appreciated by those in the art that the scope of the present invention includes those embodiments in which only one biasing spring is used.

Separating apparatus 1130 is rotatably engaged to base 1112. As in previous embodiments, separating apparatus 1130 comprises a first hair comb 1131 and a second hair comb 1132, and each is rotatable between a first position, in which the combs 1131 and 1132 engage one another, and a second position, in which the combs 1131 and 1132 are apart from one another.

A first pair of arms 1133 extends from first hair comb 1131, while a second pair of arms 1134 extends from second hair comb 1132. Each arm 1133 and 1134 comprises a guide slot 1136. A plurality of posts 1138 extend from trigger mechanism 1120, and each post 1138 engages a guide slot 1136. As seen, for example, in FIG. 31, each guide slot 1136 is formed such that, as trigger mechanism 1120 is slid to its second position, posts 1138 slide along guide slots 1136 and force each hair comb 1131 and 1132 to rotate to its respective second position.

Applicator 1110 further comprises retainer 1140, which is engaged to base 1112 and slidable between a first and a second position. As in previous embodiments, retainer 1140 retains a medicament cartridge, such as cartridge 70, on applicator 1110.

As depicted, pinion gear 1150 is engaged to base 1120. Pinion gear 1150 is engaged to first rack gear 1152 formed on trigger mechanism 1120. Pinion gear 1150 is further engaged to second rack gear 1154 formed on retainer 1140. In the depicted embodiment, rack gears 1152 and 1154 comprise two gear forms (only one is shown), but it will be appreciated by those in the art that the scope of the present invention includes those embodiments in which only one gear form is used for either first rack gear 1152, second rack gear 1154 or both gears 1152 and 1154.

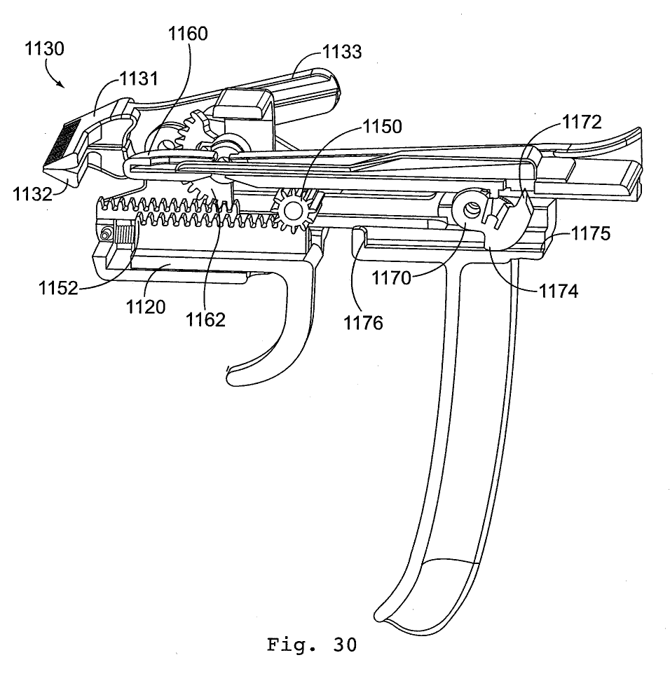

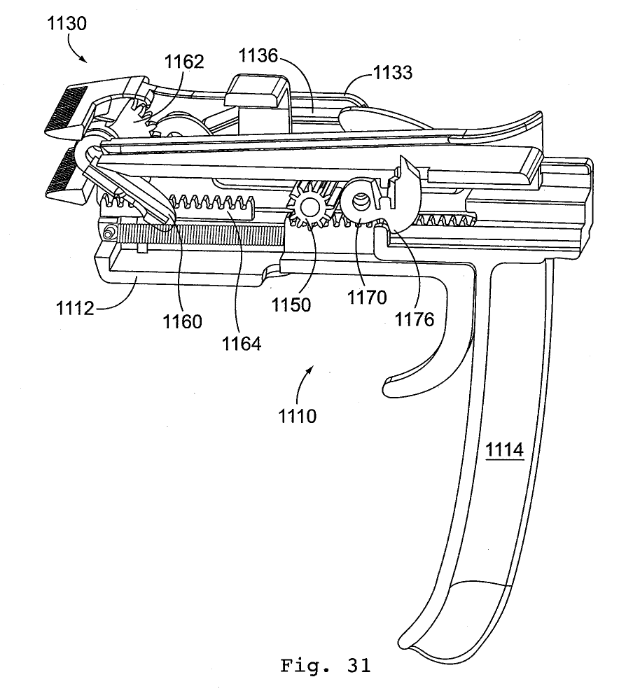

Because both trigger mechanism 1120 and retainer 1140 are engaged to pinion gear 1150, as trigger mechanism 1120 is slid to its second position, pinion gear 1150 is rotated, sliding retainer 1140 to its second position. Because first rack gear 1152 is formed on a first side of the axis of rotation of pinion gear 1150, while second rack gear 1152 is formed on a second side, opposite the first side, of the axis of rotation of pinion gear 1150, trigger mechanism 1120 will always slide in a direction that is opposite from that of retainer 1140. By way of example, in FIG. 30, as trigger mechanism 1120 is slid to the right, retainer 1140 will slide to the left.

To aid in the use of medicament cartridge 70, opening apparatus 1160 is provided. As seen, opening apparatus 1160 is positioned proximate to cracking seal 71 of medicament cartridge 70. Opening apparatus 1160 is rotatably engaged to retainer 1140, and is rotatable between a first position, in which opening apparatus 1160 is coplanar with retainer 1140, and a second position. As described in detail below, as opening apparatus 1160 is rotated to the second position, cartridge 70 is opened along cracking seal 71, allowing the medicament to exit the cartridge 70.

A pair or partial gear forms 1162 is formed on opening apparatus 1160 and are engaged to a third rack gear 1164, which is integrally formed with base 1112. In this manner, as retainer 1140 is slid to its second position, the interaction between gear forms 1162 and third rack gear 1164 will cause gear forms 1162 to rotate, thus rotating opening apparatus 1160 to its second position.

It will be understood by those of skill in the art that, while the depicted embodiment comprises a pair of partial gear forms 1162, the scope of the present invention includes those embodiments in which one or two full gears are used in lieu of gear forms 1162. The scope of the present invention also includes those embodiments in which only one gear form 1162, or full gear, is used. It will further be understood by those in the art that, while third rack gear 1164 in the depicted embodiment comprises two gear forms, the number of gear forms that comprise third rack gear may correspond to the number of gear forms 1162, or full gears, formed on opening apparatus 1160.

To aid in dispensing the medicament from cartridge 70, piercing apparatus 1170 is provided and rotatably secured to retainer 1140. In the embodiment depicted, for example, in FIG. 30, piercing apparatus 1170 comprises extension 1172 and protrusion 1174. For guidance and support, protrusion 1174 is retained in slot 1175 formed in base 1120. As retainer 1140 is slid into its second position, protrusion 1174 engages shoulder 1176, formed on base 1120. This causes piercing apparatus 1170 to rotate, and causes extension 1172 to protrude through a hole formed in retainer 1140 (not shown) and pierce cartridge 70, creating an air hole. This allows the medicament to smoothly exit cartridge 70.

While specific configurations of the invention have been described in detail, it will be appreciated by those skilled in the art that various modifications and alternatives to those details could be developed in light of the overall teachings of this disclosure. The particular arrangements disclosed herein are meant to be illustrative only and not limited as to the scope of the invention which is to be given the full breadth of the appended claims and any equivalents thereof.

What is claimed is:

1. A medicament applicator for use with a medicament cartridge, the applicator comprising:
a first handle member comprising a first end, forming a first grip portion, and a second end, forming a first brush head;
a second handle member hingedly connected to the first handle member and comprising a first end, forming a second grip portion, and a second end, forming a second brush head;
a retainer formed on the second handle member; and
an opening apparatus rotatably secured to the second handle member and engaged to the first handle member, the opening apparatus rotatable between a first position, wherein the opening apparatus is coplanar with the retainer, and a second position;
wherein when the first and second grip portions are moved proximate to each other, the first and second brush member move away from one another and the opener moves to its second position.

2. The medicament applicator as set forth in claim 1, further comprising a piercing apparatus pivotally engaged to the second handle member, the piercing apparatus comprising a first protrusion and a second protrusion and located proximate to the retainer.

3. The medicament applicator as set forth in claim 2, wherein the second protrusion of the piercing apparatus extends through a first opening formed in the retainer when the first and second grip portions are moved proximate to each other.

4. The medicament applicator as set forth in claim 3, further comprising an extension formed on the first handle member, wherein the extension engages the first protrusion of the piercing apparatus through a second hole formed in the retainer when the first grip portion and the second grip portion are moved proximate to each other.

5. The medicament applicator as set forth in claim 1, wherein the opening apparatus is engaged to the first handle member through a gear train.

6. The medicament applicator as set forth in claim 4, wherein the gear train is formed from partial gear forms.

7. The medicament applicator as set forth in claim 1, further comprising a first biasing spring engaged to the first and the second handle members, wherein the first biasing spring biases the handle members such that the first and second brush members are proximate to each other.

8. The medicament applicator as set forth in claim 1, further comprising a second biasing spring engaged to the piercing apparatus and the second handle member, wherein the second biasing spring biases the piercing apparatus such that the second protrusion does not extend through the first opening.

9. The medicament applicator as set forth in claim 1, wherein, when the medicament cartridge is engaged to the applicator, a cracking seal formed on the cartridge is positioned between the retainer and the opening apparatus.

10. The medicament applicator as set forth in claim 1, wherein, when the medicament cartridge is engaged to the applicator, the second protrusion of the piercing apparatus pierces the cartridge as it extends through the first hole.

11. A medicament applicator for use with a medicament cartridge, the medicament applicator comprising:
a base member;
a handle engaged to the base member;
a trigger mechanism engaged to the base member and movable between a first position and a second position;
a retainer engaged to the base member and movable between a first position and a second position, the retainer comprising a first hole;
a first brush head and a second brush head engaged to the base member, each brush head movable between a first position, in which the brush heads are proximate to each other, and a second position, in which the brush heads are apart from one another; and
an opening apparatus rotatably engaged to the retainer, the opening apparatus rotatable between a first position, in which the opening apparatus is coplanar with the retainer, and a second position;
wherein moving the trigger mechanism to the second position causes the retainer and the first brush head and the second brush head to move to their respective second position and causes the opening apparatus to rotate to its second position.

12. The medicament applicator as set forth in claim 11, further comprising a first plurality of arms extending from the first brush head and a second plurality of arms extending from the second brush head, wherein each arm of the first plurality of arms and the second plurality of arms comprises a guide slot.

13. The medicament applicator as set forth in claim 12, further comprising a plurality of posts extending from the trigger mechanism, wherein each post is engaged to a guide slot.

14. The medicament applicator as set forth in claim 11, further comprising a piercing apparatus engaged to the retainer and pivotable between a first position and a second position, the piercing apparatus comprising an extension the extends through the first hole when the piercing apparatus is in its second position.

15. The medicament applicator as set forth in claim 14, further comprising a protrusion formed on the piercing apparatus, wherein the protrusion engages a shoulder formed on the base member when the retainer is slid to its second position, and wherein the engagement of the protrusion with the shoulder forces the piercing apparatus to pivot to its second position.

16. The medicament applicator as set forth in claim 15, wherein the protrusion is located in a protrusion slot formed on the base member.

17. The medicament applicator as set forth in claim 14, further comprising a piercing biasing spring engaged to the piercing apparatus and the retainer, wherein the piercing biasing spring biases the piercing apparatus to its first position.

18. The medicament applicator as set forth in claim 11, further comprising a first rack gear formed on the trigger mechanism, a second rack gear formed on the retainer, and a pinion gear rotatably engaged to the base member.

19. The medicament applicator as set forth in claim 18, wherein the pinion gear is engaged to the first and the second rack gears.

20. The medicament applicator as set forth in claim 11, further comprising a first biasing spring engaged to the trigger mechanism and the base member, the first biasing spring biasing the trigger mechanism to its first position.

21. The medicament applicator as set forth in claim 11, further comprising a plurality of gears formed from the opening apparatus, wherein the plurality of gears are engaged to a third rack gear formed on the base member.

22. The medicament applicator as set forth in claim 11, further comprising a plurality of guides extending from the trigger mechanism, wherein each of the guides is engaged to one of a pair of slots formed on the base member.
